(19) United States
(12) Patent Application Publication
(10) Pub. No.: US 2025/0073428 A1
(43) Pub. Date: Mar. 6, 2025

(54) GUIDING SHEATH

(71) Applicant: TERUMO KABUSHIKI KAISHA, Tokyo (JP)

(72) Inventor: Ryo OKAMURA, Fujinomiya-shi (JP)

(73) Assignee: TERUMO KABUSHIKI KAISHA, Tokyo (JP)

(21) Appl. No.: 18/953,411

(22) Filed: Nov. 20, 2024

Related U.S. Application Data
(63) Continuation of application No. PCT/JP2023/021717, filed on Jun. 12, 2023.

(30) Foreign Application Priority Data
Jul. 14, 2022 (JP) 2022-113044

(51) Int. Cl. A61M 25/06; A61M 29/00

(52) U.S. Cl. CPC A61M 25/0662; A61M 29/00

(57) ABSTRACT

A guiding sheath, which is capable of causing a dilator and a sheath to integrally reach a carotid artery which is accessed from a radial artery and communicates with a target treatment site, and easily indwelling the sheath in the carotid artery that communicates with the target treatment site. The guiding sheath includes: a dilator in which an inner cavity, through which a guide wire is insertable is provided, over an entire length of the dilator and which includes a dilator distal end portion and a dilator main body portion; and a sheath which covers outside of the dilator and includes a sheath distal end portion and a sheath main body portion. The sheath distal end portion is disposed on a distal end side of the sheath main body portion and is formed of a material more flexible than a material forming the sheath main body portion.

### CROSS-REFERENCES TO RELATED APPLICATIONS

This application is a continuation of International Application No. PCT/JP2023/021717 filed on Jun. 12, 2023, which claims priority to Japanese Patent Application No. 2022-113044 filed on Jul. 14, 2022, the entire content of both of which is incorporated herein by reference.

### TECHNOLOGICAL FIELD

The present disclosure relates to a guiding sheath including a sheath and a dilator inserted into an inner cavity of the sheath.

### BACKGROUND DISCUSSION

When a cerebral vascular is treated via a catheter, a device usually accessed from a femoral artery is caused to reach a treatment site of the cerebral vascular via a carotid artery.

In recent years, a method of accessing a device for treatment from a radial artery has been performed (for example, see Japanese Patent Application Publication No. 2004-216176A). The method of accessing a device from a radial artery has been generally used in a coronary artery related procedure such as a percutaneous coronary intervention (PCI) for the reason that prognosis of a patient is relatively good and a period hospitalization can be shortened.

However, in a cerebral vascular treatment, it is highly difficult to cause a device accessed from a radial artery to reach a carotid artery. For example, in order for a device accessed from a right radial artery to reach a left common carotid artery, after the device reaches an aortic arch, it is necessary for the device to be rapidly curved in the aortic arch so that the device can reach the left common carotid artery, which is relatively narrower than the aortic arch. In order for the device accessed from the right radial artery to reach a right common carotid artery, it is necessary for the device to be rapidly curved in a right subclavian artery, so that the device can reach the right common carotid artery. In order for the device accessed from a left radial artery to reach the left common carotid artery, after the device reaches the aortic arch, it is necessary for the device to be rapidly curved in the aortic arch, so that the device can reach the left common carotid artery, which is relatively narrower than the aortic arch. In order for the device accessed from the left radial artery to reach the right common carotid artery, after the device reaches the aortic arch, it is necessary for the device to be rapidly curved in the aortic arch, and then after the device reaches a brachiocephalic artery, which is narrower than the aortic arch, it is necessary for the device to reach the right common carotid artery. Therefore, in the cerebral vascular treatment, there is a relatively high demand for an appropriate system for accessing a device from the radial artery to perform the cerebral vascular treatment.

### SUMMARY

A guiding sheath is disclosed, which is capable of rather easily indwelling a sheath in a carotid artery which is accessed from a radial artery and communicates with a target treatment site.

A guiding sheath according to the disclosure is a guiding sheath capable of reaching a carotid artery from a radial artery. The guiding sheath includes: a dilator in which an inner cavity, through which a guide wire is insertable is provided, over an entire length of the dilator and which includes a dilator distal end portion and a dilator main body portion; and a sheath which covers outside of the dilator and includes a sheath distal end portion and a sheath main body portion. The sheath distal end portion is disposed on a distal end side of the sheath main body portion and is formed of a material more flexible than a material forming the sheath main body portion, and at least one of the dilator distal end portion and the sheath distal end portion includes a shape-imparted portion to which a shape is imparted in advance to facilitate insertion into the carotid artery.

The guiding sheath configured as described above has a high torque transmission performance because the sheath main body portion is relatively hard, and can effectively reduce a risk of a blood vessel perforation because the sheath distal end portion is flexible. Since the guiding sheath includes the shape-imparted portion on at least one of the dilator distal end portion and the sheath distal end portion, a surgeon can integrally operate the dilator and the sheath to cause the dilator and the sheath to reach the carotid artery from the radial artery through an aortic arch or a subclavian artery. Therefore, the guiding sheath can easily indwell the sheath in the carotid artery which is accessed from the radial artery and communicates with a target treatment site.

The dilator distal end portion may be imparted with a shape in advance to facilitate insertion into the carotid artery when the dilator distal end portion is inserted into the aortic arch. Accordingly, the guiding sheath can easily reach the carotid artery from the radial artery through the aortic arch by a dilator having a shape, which is relatively easy to insert into the carotid artery.

A reinforcement body may be embedded in the sheath from the sheath distal end portion to the sheath main body portion, and a proximal end of the reinforcement body may be disposed on a distal end side of a proximal end of the sheath main body portion. Accordingly, since the reinforcement body is not disposed on a proximal end portion of the sheath main body portion, it is possible to reduce a thickness and a diameter of the proximal end portion of the sheath main body portion. Therefore, a burden on a puncture site of an arm of a patient can be reduced, and a hemostatic time can be reduced.

The reinforcement body may be a braided tube obtained by braiding a plurality of metal wires into a tubular shape, a coil obtained by spirally winding at least one metal wire, or a metal pipe in which at least one slit is formed. Accordingly, the sheath can have high kink resistance capable of withstanding strong bending.

An outer diameter of the sheath main body portion may be smaller than an outer diameter of the sheath distal end portion. Accordingly, a burden on a puncture site of an arm of a patient can be reduced, and a hemostatic time can be reduced.

The sheath main body portion may include a sheath proximal end portion disposed on a proximal end side of the sheath main body portion and a sheath physical property gradient portion disposed between the sheath proximal end portion and the sheath distal end portion, and in the sheath physical property gradient portion, a blending ratio of a material forming the sheath distal end portion and a material forming the sheath proximal end portion may gradually change from the proximal end side to the distal end side. Accordingly, the sheath physical property gradient portion whose hardness gradually decreases from the proximal end side toward the distal end side can be implemented by a stable integral structure.

The dilator main body portion may include a dilator proximal end portion disposed on a proximal end side of the dilator main body portion and a dilator physical property gradient portion disposed between the dilator proximal end portion and the dilator distal end portion, and in the dilator physical property gradient portion, a blending ratio of a material forming the dilator distal end portion and a material forming the dilator proximal end portion may gradually change from the proximal end side to the distal end side. Accordingly, the dilator physical property gradient portion whose hardness gradually decreases from the proximal end side toward the distal end side can be implemented by a stable integral structure.

The shape-imparted portion may be provided in the sheath. Accordingly, by using the sheath shape-imparted portion, the surgeon can integrally cause the sheath to reach the carotid artery from the radial artery through the aortic arch or the subclavian artery.

When the sheath and the dilator are assembled, the shape of the shape-imparted portion may be corrected to a substantially straight shape by the dilator. Accordingly, in a state where the sheath and the dilator are assembled, the surgeon can easily cause the sheath and the dilator to reach the carotid artery or the vicinity of the carotid artery.

The shape-imparted portion may include a first straight portion, a first curved portion on a distal end side of the first straight portion, a second straight portion on a distal end side of the first curved portion, and a second curved portion on a distal end side of the second straight portion, and an angle of the second straight portion with respect to the first straight portion may be 45 degrees or more. Accordingly, by using the shape-imparted portion having a specific shape of the guiding sheath, the surgeon can cause the sheath to reach the carotid artery from the radial artery through the aortic arch or the subclavian artery.

The shape of the shape-imparted portion may be a Simmons type. Accordingly, by using the Simmons type shape-imparted portion, the guiding sheath can cause the sheath to reach the carotid artery from the radial artery through the aortic arch or the subclavian artery.

A guiding sheath according to the disclosure incudes: a dilator, the dilator including an inner cavity configured to receive a guide wire, the dilator including a dilator distal end portion and a dilator main body portion; a sheath which covers outside of the dilator and includes a sheath distal end portion and a sheath main body portion; the sheath distal end portion is disposed on a distal end side of the sheath main body portion and is formed of a material more flexible than a material forming the sheath main body portion; at least one of the dilator distal end portion and the sheath distal end portion includes a shape-imparted portion; and a reinforcement body is embedded in the sheath from the sheath distal end portion to the sheath main body portion, and a proximal end of the reinforcement body is disposed on a distal end side of a proximal end of the sheath main body portion.

A guiding sheath according to the disclosure includes: a dilator, the dilator including an inner cavity configured to receive a guide wire, the dilator including a dilator distal end portion and a dilator main body portion; a sheath which covers outside of the dilator and includes a sheath distal end portion and a sheath main body portion; the sheath distal end portion is disposed on a distal end side of the sheath main body portion and is formed of a material more flexible than a material forming the sheath main body portion; at least one of the dilator distal end portion and the sheath distal end portion includes a shape-imparted portion; and a reinforcement body is embedded in the sheath from the sheath distal end portion to the sheath main body portion, and a proximal end of the reinforcement body is disposed on a distal end side of a proximal end of the sheath main body portion.

A method of indwelling a guiding sheath according to the disclosure includes: inserting the guide wire into an artery from the radial artery; inserting the guiding sheath into the artery from the radial artery while advancing the guide wire; causing the guiding sheath to reach one of an aortic arch, a right subclavian artery, or a brachiocephalic artery; pulling back the guide wire to a proximal end side with respect to the guiding sheath and partially restoring the shape of the shape-imparted portion to which the shape is imparted in advance on the at least one of the dilator distal end portion and the sheath distal end portion; causing the guiding sheath to reach one of a left common carotid artery or a right common carotid artery; and removing the guide wire and the dilator from the artery while leaving the sheath in the one of the left common carotid artery and the right common carotid artery.

### BRIEF DESCRIPTION OF THE DRAWINGS

FIG. 1 is a plan view showing a hemostatic valve and a guiding sheath according to a first embodiment in a disassembled state.

FIG. 2 is a plan view showing the hemostatic valve and the guiding sheath according to the first embodiment in an assembled state.

FIG. 3 is a plan view showing a distal end portion of a sheath of the guiding sheath according to the first embodiment.

FIG. 4 is a cross-sectional view taken along a line A-A in FIG. 3.

FIGS. 5A-5E are plan views showing a modification of a dilator shape-imparted portion, and where FIG. 5A shows a modified Simmons type, FIG. 5B shows a first type, FIG. 5C shows a Jacky type, FIG. 5D shows a second type, and FIG. 5E shows a Cobra type.

FIG. 6 is a schematic view showing a state in which the guiding sheath according to the first embodiment is caused to reach a left common carotid artery from a right radial artery.

FIG. 7 is a plan view showing an example in which the guiding sheath according to the first embodiment and a Y connector are used.

FIG. 8 is a plan view showing a sheath of a guiding sheath according to a second embodiment in a disassembled state and a distal end portion of a dilator.

FIG. 9 is a plan view showing a hemostatic valve and a guiding sheath according to a third embodiment in a disassembled state.

FIGS. 10A-10C are plan views showing the hemostatic valve and the guiding sheath according to the third embodiment in an assembled state, and where

FIG. 10A shows the third embodiment, FIG. 10B shows a modification, and FIG. 10C shows another modification.

FIG. 11 is Table 1 with different dilator shaped-imparted portions.

### DETAILED DESCRIPTION

Set forth below with reference to the accompanying drawings is a detailed description of embodiments of a guiding sheath including a sheath and a dilator inserted into an inner cavity of the sheath. A dimensional ratio in the drawings may be exaggerated for convenience of illustration and may be different from an actual ratio. In the following description, a side of a guiding sheath 10 to be operated by a surgeon is referred to as a “proximal end side”, and a side to be inserted into a body is referred to as a “distal end side”.

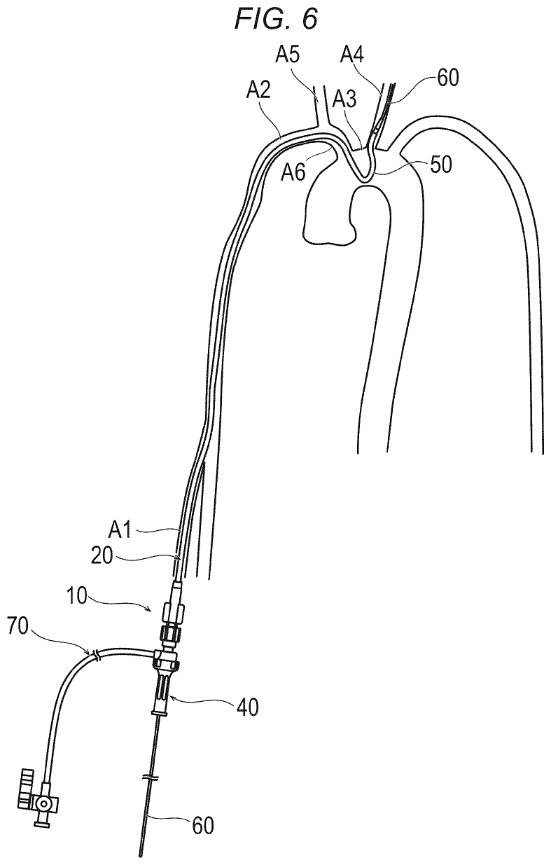

As shown in FIG. 6, the guiding sheath 10 according to a first embodiment of the disclosure is used to access a device for treating a cerebral vascular from a radial artery and cause the device to reach the cerebral vascular. That is, the guiding sheath 10 can be used by a surgeon accessing from a right radial artery A1 or a left radial artery to reach a right common carotid artery A5 or a left common carotid artery A4.

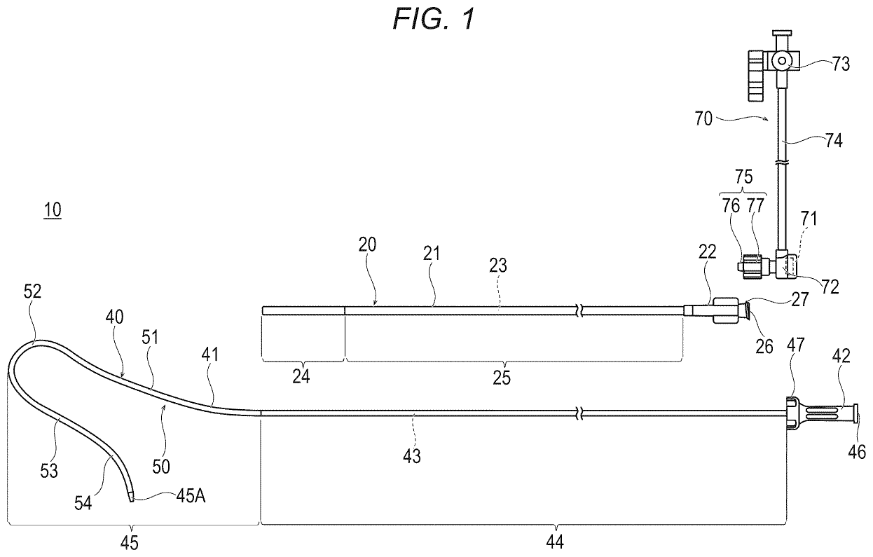

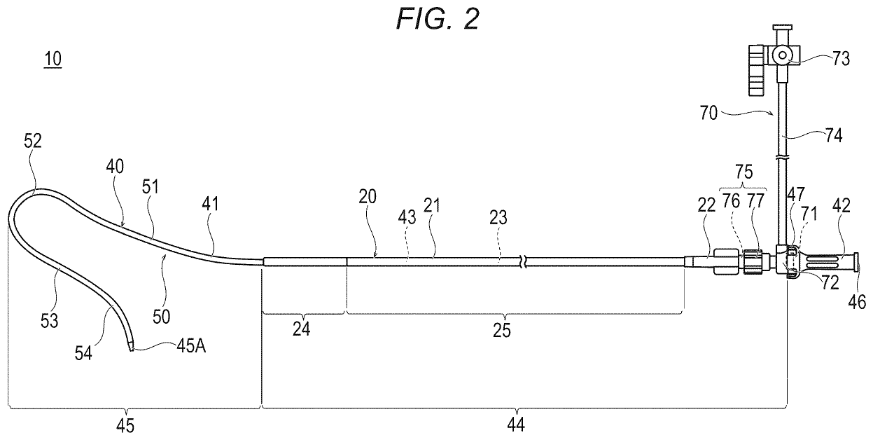

As shown in FIGS. 1 and 2, the guiding sheath 10 can include a sheath 20 that provides a sheath inner cavity 23 serving as a passage for a treatment device, and a dilator 40 that functions as a core for inserting the sheath 20 to a target position.

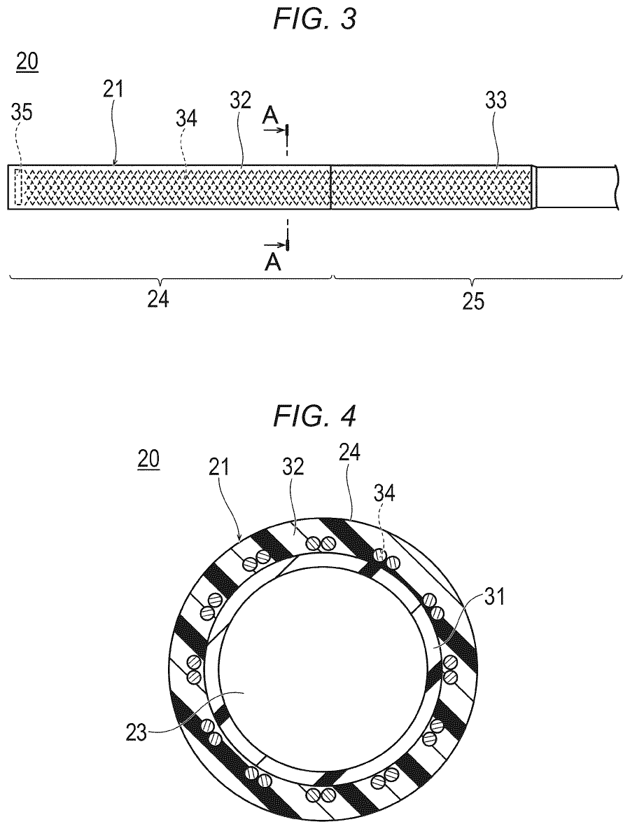

As shown in FIGS. 1 to 4, the sheath 20 can include a flexible tubular sheath tube body 21 and a sheath hub 22 fixed to a proximal end of the sheath tube body 21. The sheath tube body 21 is implemented by a tubular body having flexibility, and the sheath inner cavity 23 is formed over the entire length in a substantially central portion of the sheath tube body 21.

The sheath tube body 21 includes an inner layer 31 in which the sheath inner cavity 23 is formed, a distal end side outer layer 32 covering an outer peripheral surface of the inner layer 31 on a distal end portion of the sheath tube body 21, a main body outer layer 33 covering the outer peripheral surface of the inner layer 31 on a proximal end side of the distal end side outer layer 32, a reinforcement body 34 embedded in the distal end side outer layer 32 and the main body outer layer 33, and a radiopaque marker 35. An outer surface of the distal end portion of the sheath tube body 21 is preferably coated with a hydrophilic coat.

The inner layer 31 is formed of a resin material. The resin material of the inner layer 31 is preferably a fluorine-based resin such as polytetrafluoroethylene (PTFE), a low-friction material such as high-density polyethylene (HDPE), or the like. A polyamide resin, a polyamide elastomer or a polyester, a polyester elastomer, or the like may be used.

The distal end side outer layer 32 and the main body outer layer 33 are formed of a resin material. The resin material of the distal end side outer layer 32 is more flexible than the material of the main body outer layer 33.

The reinforcement body 34 is a braided tube obtained by braiding a plurality of metal wires into a tubular shape, a coil obtained by spirally winding at least one metal wire, or a metal pipe in which at least one slit is formed.

The sheath tube body 21 can include a sheath distal end portion 24 on the distal end side and a sheath main body portion 25 disposed on the proximal end side of the sheath distal end portion 24. The sheath distal end portion 24 is formed by the inner layer 31, the reinforcement body 34, and the distal end side outer layer 32. The sheath main body portion 25 is formed by the inner layer 31, the reinforcement body 34, and the main body outer layer 33. The most distal end of the reinforcement body 34 is disposed at a position close to the most distal end of the sheath distal end portion 24, and the most proximal end of the reinforcement body 34 is disposed on the sheath main body portion 25 at a position on the proximal end side of about 20 cm to 30 cm from the most distal end of the sheath tube body 21. Accordingly, a distal end portion of the sheath main body portion 25 is formed by the inner layer 31, the reinforcement body 34, and the main body outer layer 33, and a proximal end portion of the sheath main body portion 25 is formed by the inner layer 31 and the main body outer layer 33. Accordingly, an outer diameter of the distal end portion of the sheath main body portion 25 including the reinforcement body 34 is similar to an outer diameter of the sheath distal end portion 24 including the reinforcement body 34. Since the proximal end portion of the sheath main body portion 25 that does not include the reinforcement body 34 is thinned by an amount of the reinforcement body 34 that is not provided, an outer diameter of the proximal end portion of the sheath main body portion 25 that does not include the reinforcement body 34 is smaller than the outer diameter of the distal end portion of the sheath main body portion 25 that includes the reinforcement body 34. Therefore, a burden on a puncture site of an arm of a patient during a procedure can be reduced, and a hemostatic time can be reduced.

Since the main body outer layer 33 of the sheath main body portion 25 is formed of a material harder than that of the distal end side outer layer 32 of the sheath distal end portion 24, the sheath main body portion 25 has a high torque transmission performance. Since the distal end side outer layer 32 of the sheath distal end portion 24 is formed of a material more flexible than that of the main body outer layer 33 of the sheath main body portion 25, the sheath distal end portion 24 has high flexibility. Further, since the sheath distal end portion 24 includes the reinforcement body 34, the sheath distal end portion 24 has high kink resistance capable of withstanding strong bending.

The material of the distal end side outer layer 32 and the main body outer layer 33 is not particularly limited, and examples of the material of distal end side outer layer 32 and the main body outer layer 33 can include polyethylene, polyester elastomer, and urethane. The sheath 20 may not include the reinforcement body 34. An outer diameter of the sheath tube body 21 is preferably, for example, 4 Fr or 5 Fr.

The sheath hub 22 is fixed to the proximal end of the sheath tube body 21. The sheath hub 22 has an inner cavity communicating with the sheath inner cavity 23, and opens at a sheath hub opening portion 26 on the proximal end side. A male screw 27 is formed on an outer peripheral surface of a proximal end portion of the sheath hub 22. The male screw 27 can be connected to a hemostatic valve 70 and a Y connector 80 described later.

The dilator 40 includes a dilator tube body 41 that can be inserted into the sheath tube body 21, and a dilator hub 42 fixed to a proximal end of the dilator tube body 41. A dilator inner cavity 43 is formed in a central portion of the dilator tube body 41 along an axis extending from a distal end to a proximal end of the dilator 40. The dilator tube body 41 includes a tubular dilator main body portion 44 and a dilator distal end portion 45 located on a distal end side of the dilator main body portion 44.

The dilator main body portion 44 preferably has a substantially constant outer diameter along the axis, but is not limited to having a substantially constant outer diameter along the axis. An outer peripheral surface of the dilator main body portion 44 is preferably circular in a cross section orthogonal to the axis, but is not limited to being circular in a cross section orthogonal to the axis. By disposing the dilator tube body 41 in the sheath tube body 21, the outer peripheral surface of the dilator main body portion 44 is in contact with an inner peripheral surface of the sheath tube body 21 with substantially no gap between the outer peripheral surface of the dilator main body portion 44 and the inner peripheral surface of the sheath tube body 21 or is adjacent to the inner peripheral surface of the sheath tube body 21 with a minute gap the outer peripheral surface of the dilator main body portion 44 and the inner peripheral surface of the sheath tube body 21. Therefore, an outer diameter of the dilator main body portion 44 is substantially equal to an inner diameter of the sheath tube body 21 or slightly smaller than the inner diameter of the sheath tube body 21.

The dilator distal end portion 45 is formed in a tubular shape by a resin that is more flexible than the resin forming the dilator main body portion 44. An outer peripheral surface of the dilator distal end portion 45 is preferably circular in a cross section orthogonal to the axis, but is not limited to being circular in a cross section orthogonal to the axis. An outer diameter of a proximal end portion of the dilator distal end portion 45 is preferably equal to the outer diameter of the dilator main body portion 44. Accordingly, outer surfaces of the dilator distal end portion 45 and the dilator main body portion 44 can be smoothly connected to each other without any step. The dilator distal end portion 45 includes a dilator shape-imparted portion 50 to which a specific shape is imparted in advance. The outer surface of the dilator distal end portion 45 is preferably coated with a hydrophilic coat. The dilator distal end portion 45 (dilator shape-imparted portion 50) includes a tapered portion 45A at the most distal end, and an outer diameter of the tapered portion 45A gradually decreases toward the distal end side.

A shape of the dilator shape-imparted portion 50 can be, for example, a Simmons® type. As shown in Table 1 (FIG. 11), the Simmons type dilator shape-imparted portion 50 includes a first straight portion 51, a curved first curved portion 52 on the distal end side of the first straight portion 51, a straight second straight portion 53 on the distal end side of the first curved portion 52, and a second curved portion 54 on the distal end side of the second straight portion 53. As shown in Table 1, an angle of the second straight portion 53 with respect to the first straight portion 51 can be, for example, 180 degrees or more, and the second curved portion 54 is curved in a direction opposite to the first curved portion 52.

Materials of the dilator distal end portion 45 and the dilator main body portion 44 are, for example, high-density polyethylene, low-density polyethylene, vinyl chloride, and urethane.

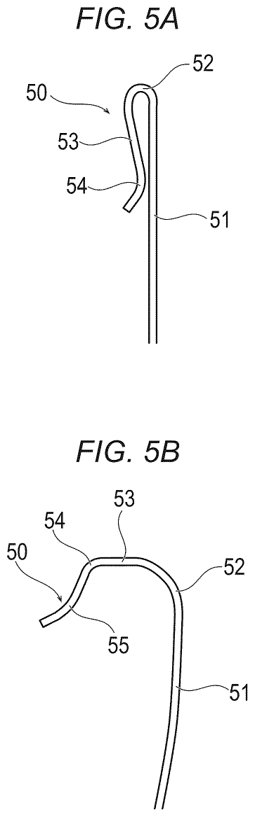

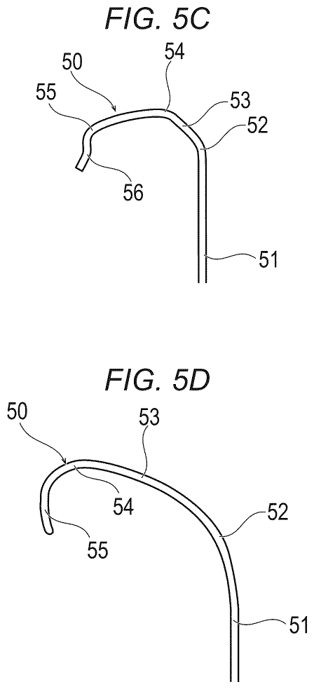

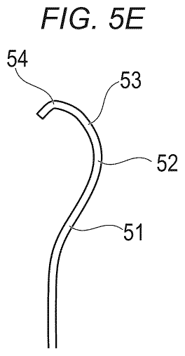

The shape of the dilator shape-imparted portion 50 is not particularly limited, and may be, for example, a modified Simmons® type shown in FIG. 5A, a first type shown in FIG. 5B, a Jacky® type shown in FIG. 5C, a second type shown in FIG. 5D, or a Cobra® type shown in FIG. 5E.

As shown in FIG. 5A, the modified Simmons type dilator shape-imparted portion 50 includes, as shown in Table 1 (FIG. 11), the first straight portion 51, the first curved portion 52 on the distal end side of the first straight portion 51, the second straight portion 53 on the distal end side of the first curved portion 52, the second curved portion 54 on the distal end side of the second straight portion 53, and a third curved portion 55 on the distal end side of the second curved portion 54. An angle of the second straight portion 53 with respect to the first straight portion 51 can be, for example, 180 degrees or more, and the second curved portion 54 is curved in a direction opposite to the first curved portion 52.

As shown in FIG. 5B, the first type dilator shape-imparted portion 50 includes the first straight portion 51, the first curved portion 52 on the distal end side of the first straight portion 51, the second straight portion 53 on the distal end side of the first curved portion 52, the second curved portion 54 on the distal end side of the second straight portion 53, and the third curved portion 55 on the distal end side of the second curved portion 54. An angle of the second straight portion 53 with respect to the first straight portion 51 can be, for example, 90 degrees or more, the second curved portion 54 is curved in the same direction as the first curved portion 52, and the third curved portion 55 is curved in a direction opposite to the first curved portion 52.

As shown in FIG. 5C, the Jacky type dilator shape-imparted portion 50 includes the first straight portion 51, the first curved portion 52 on the distal end side of the first straight portion 51, the second straight portion 53 on the distal end side of the first curved portion 52, the second curved portion 54 on the distal end side of the second straight portion 53, the third curved portion 55 on the distal end side of the second curved portion 54, and a fourth curved portion on the distal end side of the third curved portion 55. The angle of the second straight portion 53 with respect to the first straight portion 51 can be, for example, 45 degrees or more, the second curved portion 54 is curved in the same direction as the first curved portion 52, the third curved portion 55 is curved in the same direction as the first curved portion 52, and the fourth curved portion is curved in a direction opposite to the first curved portion 52.

As shown in FIG. 5D, the second type dilator shape-imparted portion 50 includes the first straight portion 51, the first curved portion 52 on the distal end side of the first straight portion 51, the second straight portion 53 on the distal end side of the first curved portion 52, the second curved portion 54 on the distal end side of the second straight portion 53, the third curved portion 55 on the distal end side of the second curved portion 54, and the fourth curved portion on the distal end side of the third curved portion 55. The angle of the second straight portion 53 with respect to the first straight portion 51 can be, for example, 90 degrees or more, the second curved portion 54 is curved in the same direction as the first curved portion 52, and the third curved portion 55 is curved in the same direction as the first curved portion 52.

As shown in FIG. 5E, the cobra type dilator shape-imparted portion 50 includes the first straight portion 51, the first curved portion 52 on the distal end side of the first straight portion 51, the second straight portion 53 on the distal end side of the first curved portion 52, and the second curved portion 54 on the distal end side of the second straight portion 53. The angle of the second straight portion 53 with respect to the first straight portion 51 can be, for example, 90 degrees or more, and the second curved portion 54 is curved in the same direction as the first curved portion 52.

As shown in FIGS. 1 and 2, the dilator hub 42 is fixed to the proximal end of the dilator tube body 41. The dilator hub 42 has an inner cavity communicating with the dilator inner cavity 43, and opens at a dilator hub opening portion 46 on the proximal end side. The dilator hub 42 includes a plurality of coupling claws 47 on the distal end side that can be coupled to an outer peripheral surface of the hemostatic valve 70 or the Y connector 80, which will be described later, so as to be hooked on the hemostatic valve 70 or the Y connector 80.

The hemostatic valve 70 includes a valve body 71, a housing 72 accommodating the valve body 71, a three-way stopcock 73 capable of opening and closing and switching a flow path, and a side tube 74 connecting the housing 72 and the three-way stopcock 73. The valve body 71 is a backflow prevention valve for preventing the outflow of blood, and a cut hole that can be opened by deformation is formed in an elastically deformable disk-shaped member. The housing 72 accommodates the valve body 71 on one end side of a through-hole, and includes a connector 75 that can be coupled to the sheath hub 22 on the other end side. The connector 75 includes a coupling tube portion 76 having an outer peripheral surface capable of entering the sheath hub opening portion 26 and coming into close contact with an inner peripheral surface of the sheath hub opening portion 26, and a rotation connector 77 rotatable around an outer periphery of the coupling tube portion 76. A female screw that can be screwed with the male screw 27 of the sheath hub 22 is formed on an inner peripheral surface of the rotation connector 77. The side tube 74 connects an inner cavity of the housing 72 on the distal end side of the valve body 71 and the three-way stopcock 73.

Next, an example of a method of using the guiding sheath 10 according to the first embodiment will be described. Here, as shown in FIG. 6, a case where the guiding sheath 10 is inserted from the right radial artery A1 and indwelt in the left common carotid artery A4 will be described as an example. The surgeon may use the guiding sheath 10 to cause the guiding sheath 10 accessed from the right radial artery A1 to reach the right common carotid artery A5, or may use the guiding sheath 10 to cause the guiding sheath 10 accessed from the left radial artery to reach the left common carotid artery A4 or the right common carotid artery A5.

First, before the guiding sheath 10 is introduced into the blood vessel, the sheath 20, the hemostatic valve 70, and the dilator 40 are coupled to form an assembled state as shown in FIGS. 1 and 2. At the time of coupling, the coupling tube portion 76 of the hemostatic valve 70 is inserted into the sheath hub opening portion 26, the rotation connector 77 is rotated, and the female screw of the rotation connector 77 is screwed to the male screw 27 of the sheath hub 22. Accordingly, the sheath 20 and the hemostatic valve 70 are coupled to each other. Next, the dilator 40 is inserted into the housing 72 of the hemostatic valve 70, and inserted into the sheath 20 by opening and passing the valve body 71 in the housing 72. The dilator distal end portion 45 including the dilator shape-imparted portion 50 passes through the hemostatic valve 70 and the sheath 20, and protrudes further toward the distal end side of the sheath 20. Then, the coupling claws 47 of the dilator hub 42 are coupled to the housing 72 of the hemostatic valve 70 so as to be hooked on the housing 72 of the hemostatic valve 70. Accordingly, the sheath 20, the hemostatic valve 70, and the dilator 40 are coupled to each other. Therefore, when the guiding sheath 10 is inserted into the blood vessel, the sheath 20, the hemostatic valve 70, and the dilator 40 can be integrally operated.

Next, the surgeon punctures the radial artery A1 by a known method, inserts a short sheath, and performs pre-dilation. Next, a guide wire 60 is inserted into the blood vessel through the short sheath, and the short sheath is removed. Subsequently, the guide wire 60 is advanced, and the guiding sheath 10 is inserted into the blood vessel along the guide wire 60. The dilator shape-imparted portion 50 of the dilator distal end portion 45 has a straight shape extending straight due to the rigidity of the guide wire 60 so as to be rather easily inserted into the blood vessel.

Next, the surgeon causes the guiding sheath 10 in the assembled state to reach an aortic arch A3 from a right subclavian artery A2. At this time, the surgeon keeps the guide wire 60 in the vicinity of an ascending aorta such that a blood vessel perforation does not occur. When the guiding sheath 10 reaches the meandering portion of the ascending aorta, the surgeon slightly pulls back the guide wire 60. Accordingly, a flexible distal end portion of the guide wire 60 is located inside the dilator shape-imparted portion 50, and the shape of the dilator shape-imparted portion 50 can be restored. Next, the surgeon applies a torque to the guiding sheath 10 to engage the dilator shape-imparted portion 50 with the left common carotid artery A4.

Next, the surgeon advances the guiding sheath 10 along the left common carotid artery A4. When a distal end of the guiding sheath 10 reaches the vicinity of a target, the surgeon stops pushing the guiding sheath 10. Next, the surgeon separates the coupling claws 47 of the dilator hub 42 from the housing 72 of the hemostatic valve 70 to release the coupling between the hemostatic valve 70 and the dilator 40. Thereafter, the surgeon leaves the sheath 20 in the blood vessel, and removes the guide wire 60 and the dilator 40. When the dilator 40 is removed, the valve body 71 in the housing 72 of the hemostatic valve 70 is closed to prevent backflow of blood. Accordingly, the sheath 20 and the hemostatic valve 70 can be used to perform a procedure of inserting a medical instrument corresponding to a treatment site to perform diagnosis or treatment.

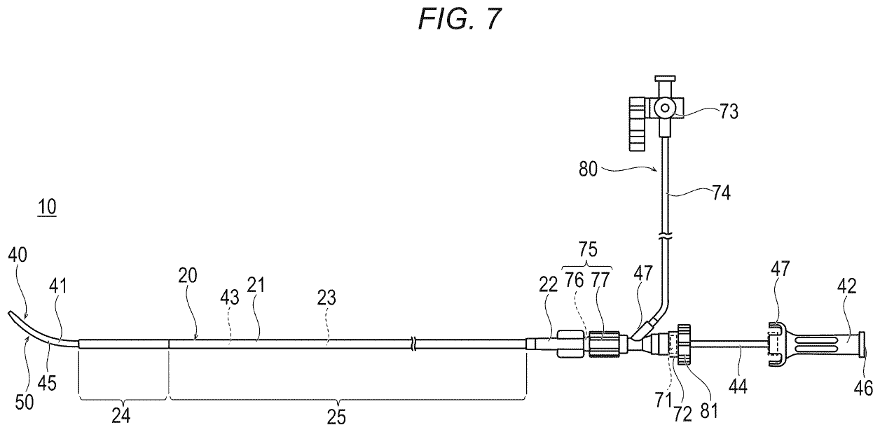

Next, another example of the method of using the guiding sheath 10 according to the first embodiment will be described. Here, as shown in FIG. 7, the Y connector 80 is used instead of the hemostatic valve 70.

Similar to the hemostatic valve 70 described above, the Y connector 80 includes the valve body 71, the housing 72 accommodating the valve body 71, the three-way stopcock 73 capable of opening and closing and switching a flow path, and the side tube 74 connecting the housing 72 and the three-way stopcock 73. The housing 72 includes a hemostatic valve 81 on one end side of a through-hole, and includes the connector 75 that can be coupled to the sheath hub 22 on the other end side. The connector 75 includes the coupling tube portion 76 and the rotation connector 77. The valve body 71 is disposed inside the hemostatic valve 81. When the hemostatic valve 81 is rotated clockwise, a compressive force acts on the valve body 71 to close a hole of the valve body 71. When the hemostatic valve 81 is rotated counterclockwise, the compressive force acting on the valve body 71 is eliminated, and the hole of the valve body 71 can be widened.

First, before the guiding sheath 10 is introduced into the blood vessel, as shown in FIG. 7, the sheath 20, the Y connector 80, and the dilator 40 are coupled to form an assembled state. At the time of coupling, the coupling tube portion 76 of the connector 80 is inserted into the sheath hub opening portion 26, the rotation connector 77 is rotated, and the female screw of the rotation connector 77 is screwed to the male screw 27 of the sheath hub 22. Accordingly, the sheath 20 and the Y connector 80 are coupled to each other. Next, the hemostatic valve 81 in which the valve body 71 is disposed is rotated counterclockwise at a proximal end portion of the Y connector 80. Accordingly, the compressive force acting on the valve body 71 is eliminated, and the hole of the valve body 71 can be widened. Next, the surgeon inserts the dilator 40 into the hemostatic valve 81 of the Y connector 80. The dilator 40 passes through the valve body 71 in the hemostatic valve 81 while opening, and reaches the sheath 20. The surgeon causes only a part of the dilator shape-imparted portion 50 to protrude toward the distal end side of the sheath 20. The dilator shape-imparted portion 50 disposed inside the sheath 20 is held to be deformed straight due to the rigidity of the sheath 20. Next, the surgeon rotates the hemostatic valve 81 clockwise to apply a compressive force to the valve body 71. Accordingly, the valve body 71 is closed, and the dilator 40 is fixed to the hemostatic valve 81 of the Y connector 80. Accordingly, the sheath 20, the Y connector 80, and the dilator 40 are coupled to each other. Therefore, when the guiding sheath 10 is inserted into the blood vessel, the sheath 20, the Y connector 80, and the dilator 40 can be integrally operated. At this time, the dilator shape-imparted portion 50 is held in a state in which only a part on the distal end side protrudes from the sheath 20 and the other part is linearly deformed inside the sheath 20.

Next, the surgeon punctures the radial artery A1 by a known method, inserts a short sheath, and performs pre-dilation. Next, the guide wire 60 is inserted into the blood vessel through the short sheath, and the short sheath is removed. Subsequently, the guide wire 60 is advanced, and the guiding sheath 10 is inserted into the blood vessel along the guide wire 60. The dilator shape-imparted portion 50 of the dilator distal end portion 45 has a linear shape due to the rigidity of the guide wire 60 so as to be easily inserted into the blood vessel.

Next, the surgeon causes the guiding sheath 10 in the assembled state to reach the aortic arch A3 from the right subclavian artery A2. At this time, the surgeon keeps the guide wire 60 in the vicinity of the ascending aorta such that a blood vessel perforation does not occur. When the guiding sheath 10 reaches the meandering portion of the ascending aorta, the surgeon slightly pulls back the guide wire 60. Accordingly, the flexible distal end portion of the guide wire 60 is located inside the dilator shape-imparted portion 50 of the dilator 40. Next, after releasing the hemostatic valve 81 of the Y connector 80, the surgeon pushes the dilator 40 and the guide wire 60 forward until the entire dilator shape-imparted portion 50 protrudes from a distal end of the sheath 20. Accordingly, the dilator shape-imparted portion 50 restores the original shape. Next, the surgeon applies a torque to the guiding sheath 10 to engage the shape-imparted portion with the left common carotid artery A4.

Next, the surgeon advances the guiding sheath 10 along the left common carotid artery A4. When the distal end of the guiding sheath 10 reaches the vicinity of a target, the surgeon stops pushing the guiding sheath 10. Next, the surgeon releases the hemostatic valve 81, leaves the sheath 20 in the blood vessel, and removes the guide wire 60 and the dilator 40. Accordingly, the sheath 20 and the Y connector 80 can be used to perform a procedure of inserting a medical instrument corresponding to a treatment site to perform diagnosis or treatment.

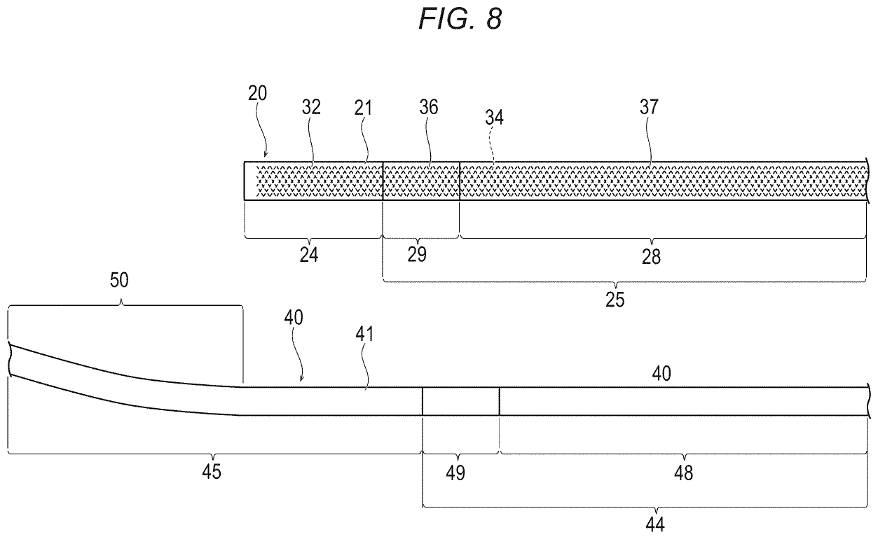

As shown in FIG. 8, the guiding sheath 10 according to a second embodiment is different from that of the first embodiment in that physical properties of the sheath tube body 21 and the dilator tube body 41 gradually change along an axial direction.

The sheath tube body 21 includes the sheath distal end portion 24 on the distal end side and the sheath main body portion 25 disposed on the proximal end side of the sheath distal end portion 24. The sheath main body portion 25 includes a sheath proximal end portion 28 having a uniform hardness along the axis, and a sheath physical property gradient portion 29 disposed on the distal end side of the sheath proximal end portion 28 and on the proximal end side of the sheath distal end portion 24. The sheath physical property gradient portion 29 is formed such that the hardness gradually decreases from the proximal end side toward the distal end side.

The most distal end of the reinforcement body 34 is disposed at a position close to the most distal end of the sheath distal end portion 24, and the most proximal end of the reinforcement body 34 is disposed at a position close to the most distal end of the sheath proximal end portion 28. Accordingly, the reinforcement body 34 is disposed over substantially the entire length of the sheath tube body 21. The reinforcement body 34 can be, for example, a flat plate-shaped coil. The outer diameter of the sheath tube body 21 is constant over substantially the entire length of the sheath tube body 21. The inner layer 31 is common to the sheath distal end portion 24 and the sheath main body portion 25.

The outer layer has different hardness in the sheath distal end portion 24, the sheath physical property gradient portion 29, and the sheath proximal end portion 28. The outer layer includes the distal end side outer layer 32 disposed in the sheath distal end portion 24, a gradient portion outer layer 36 disposed in the sheath physical property gradient portion 29, and a proximal end side outer layer 37 disposed in the sheath proximal end portion 28. The hardness of the distal end side outer layer 32 is substantially constant along the axial direction, and the hardness of the proximal end side outer layer 37 is also substantially constant along the axial direction. The hardness of the gradient portion outer layer 36 gradually decreases from a proximal end toward a distal end so as to match the hardness of the proximal end side outer layer 37 at the proximal end and match the hardness of the distal end side outer layer 32 at the distal end. For such a configuration, a material of the proximal end side outer layer 37 is harder than a material of the distal end side outer layer 32, and a material of the gradient portion outer layer 36 is a mixture of the material of the proximal end side outer layer 37 and the material of the distal end side outer layer 32. In the gradient portion outer layer 36, it is preferable that a blending of a resin material on the proximal end side and a resin on the distal end side gradually changes from the proximal end side to the distal end side. For example, the resin on the proximal end side is high-density polyethylene, and the resin on the distal end side is low-density polyethylene. Since the resin on the proximal end side and the resin on the distal end side are materials having different hardness and the same composition, the resins can be mixed with high compatibility. Accordingly, when the outer layer is molded, the gradient portion outer layer 36 in which the blending of the resin on the proximal end side and the resin on the distal end side gradually changes can be molded by extruding the material while gradually changing the blending of the resin on the proximal end side and the resin on the distal end side by extrusion molding. In the extrusion molding, when the resin on the proximal end side and the resin on the distal end side are mixed and extruded by different screws, the blending of the resin on the proximal end side and the resin on the distal end side can be gradually changed by adjusting rotation speeds of the screws. The resin on the proximal end side and the resin on the distal end side may not be materials having the same composition as long as they can be mixed. The resin on the proximal end side and the resin on the distal end side are not limited to polyethylene as long as they are resins, and may be, for example, vinyl chloride or urethane. In addition, the gradient portion outer layer 36 may not have a structure in which the blending of the resin on the proximal end side and the resin on the distal end side gradually changes from the proximal end side to the distal end side. For example, the gradient portion outer layer 36 may be formed by overlapping, in a radial direction, a first layer formed of the resin on the proximal end side that becomes thinner gradually toward a distal end direction and a second layer formed of the resin on the distal end side that becomes thinner gradually toward a proximal end direction such that the rigidity gradually changes.

The dilator tube body 41 includes the dilator distal end portion 45 on the distal end side and the dilator main body portion 44 disposed on the proximal end side of the dilator distal end portion 45. The dilator main body portion 44 includes a dilator proximal end portion 48 having a uniform hardness along the axis, and a dilator physical property gradient portion 49 disposed on the distal end side of the dilator proximal end portion 48 and on the proximal end side of the dilator distal end portion 45.

The hardness of the dilator distal end portion 45 is substantially constant along the axial direction except for the tapered portion 45A, and the hardness of the dilator proximal end portion 48 is also substantially constant along the axial direction. The hardness of the dilator physical property gradient portion 49 gradually decreases from a proximal end toward a distal end so as to match the hardness of the dilator proximal end portion 48 at the proximal end and to match the hardness of the outer layer of the dilator distal end portion 45 at the distal end. For such a configuration, a material of the dilator proximal end portion 48 is harder than a material of the dilator distal end portion 45, and a material of the dilator physical property gradient portion 49 is a mixture of the material of the dilator distal end portion 45 and the material of the dilator proximal end portion 48. It is preferable that a blending of the material of the dilator physical property gradient portion 49 gradually changes from the proximal end side to the distal end side. The dilator physical property gradient portion 49 can be formed by the same method as the above-described sheath physical property gradient portion 29 (or the gradient portion outer layer 36). An outer diameter of the dilator tube body 41 is constant over substantially the entire length of the dilator tube body 41 except for the tapered portion 45A.

The distal end of the dilator physical property gradient portion 49 is located on the proximal end side with respect to a distal end of the sheath physical property gradient portion 29, and is located on the distal end side with respect to a proximal end of the sheath physical property gradient portion 29. The proximal end of the dilator physical property gradient portion 49 is located on the proximal end side with respect to the proximal end of the sheath physical property gradient portion 29.

A method of using the guiding sheath 10 according to the second embodiment is the same as that of the first embodiment. When the sheath 20, the hemostatic valve 70 (or the Y connector 80), and the dilator 40 are coupled to form an assembled state, the dilator shape-imparted portion 50 protrudes from the sheath 20, and a proximal end portion of the sheath physical property gradient portion 29 partially overlaps a distal end portion of the dilator physical property gradient portion 49.

Since the dilator 40 is provided with the dilator physical property gradient portion 49, a distal end portion of the dilator 40 does not become too hard because the flexibility gradually increases toward the distal end side, and a risk of a blood vessel perforation can be reduced. In addition, since the sheath 20 includes the sheath physical property gradient portion 29, a distal end portion of the sheath 20 is flexible and has high followability to blood vessels. Therefore, when the guiding sheath 10 passes a large meandering portion, a gap is hardly generated between the dilator 40 and the sheath 20, and the guiding sheath 10 can smoothly pass the meandering portion.

In the guiding sheath 10, the dilator physical property gradient portion 49 is provided on the dilator 40, the sheath physical property gradient portion 29 is provided on the sheath 20, and the positions of the dilator physical property gradient portion 49 and the sheath physical property gradient portion 29 are shifted in the axial direction. Therefore, the guiding sheath 10 as a whole including the sheath 20 and the dilator 40 has sufficient rigidity on a proximal end portion, and the rigidity gradually decreases from the proximal end side toward the distal end side.

Accordingly, since the guiding sheath 10 has sufficient rigidity on the proximal end portion of the guiding sheath 10, the guiding sheath 10 has a high torque transmission performance and can easily orient a distal end portion. Further, the distal end portion of the guiding sheath 10 does not become too hard, and the risk of the blood vessel perforation can be reduced.

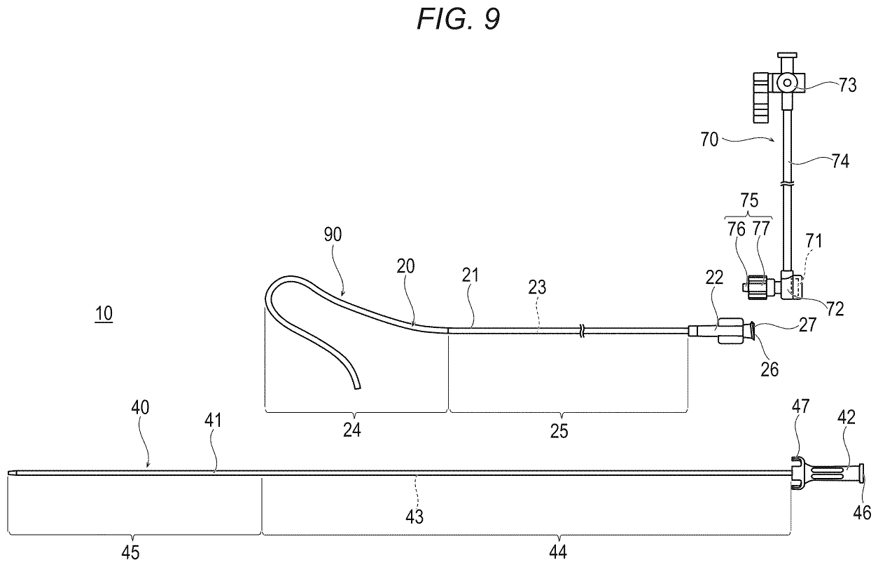

As shown in FIG. 9, the guiding sheath 10 according to the third embodiment is different from the first embodiment in that the distal end portion of the sheath tube body 21 is shaped instead of the dilator tube body 41.

The sheath tube body 21 includes a sheath shape-imparted portion 90, to which a specific shape is imparted in advance, on the sheath distal end portion 24 that is disposed on the distal end side of the tubular sheath main body portion 25 and is more flexible than the sheath main body portion 25. Similar to the dilator shape-imparted portion 50 of the first embodiment, the sheath shape-imparted portion 90 can be of a Simmons type or various types shown in FIGS. 5A to 5E. A specific layer structure of the sheath tube body 21 may be the same as or different from that of the first embodiment or the second embodiment.

As shown in FIG. 9, the dilator tube body 41 includes the tubular dilator main body portion 44 and the dilator distal end portion 45 which is more flexible than the dilator main body portion 44 located on the distal end side of the dilator main body portion 44. The dilator tube body 41 is not shaped and is formed straight. The dilator tube body 41 is the same as in the first embodiment and the second embodiment except that the dilator tube body 41 is not shaped, and may be different from the first embodiment or the second embodiment.

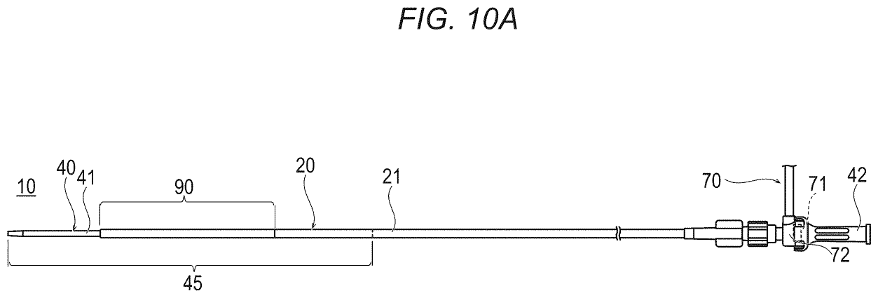

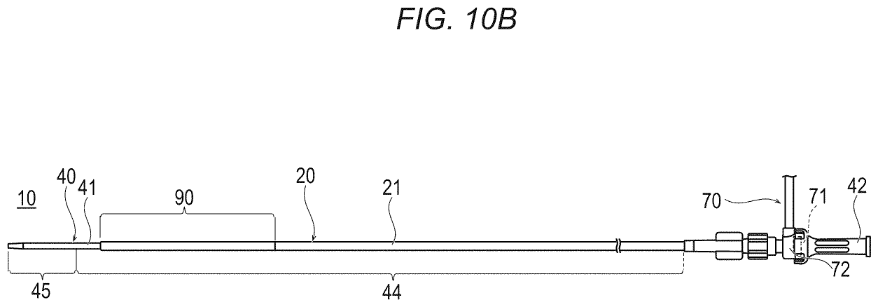

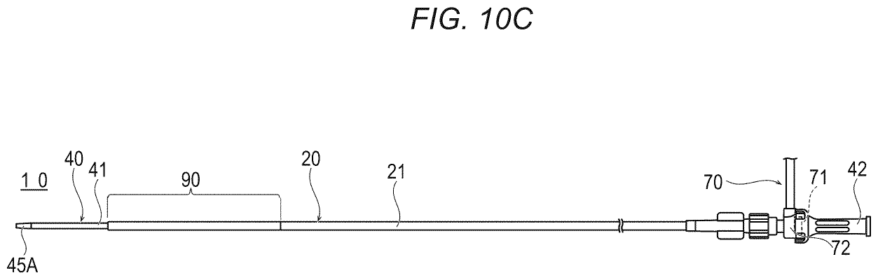

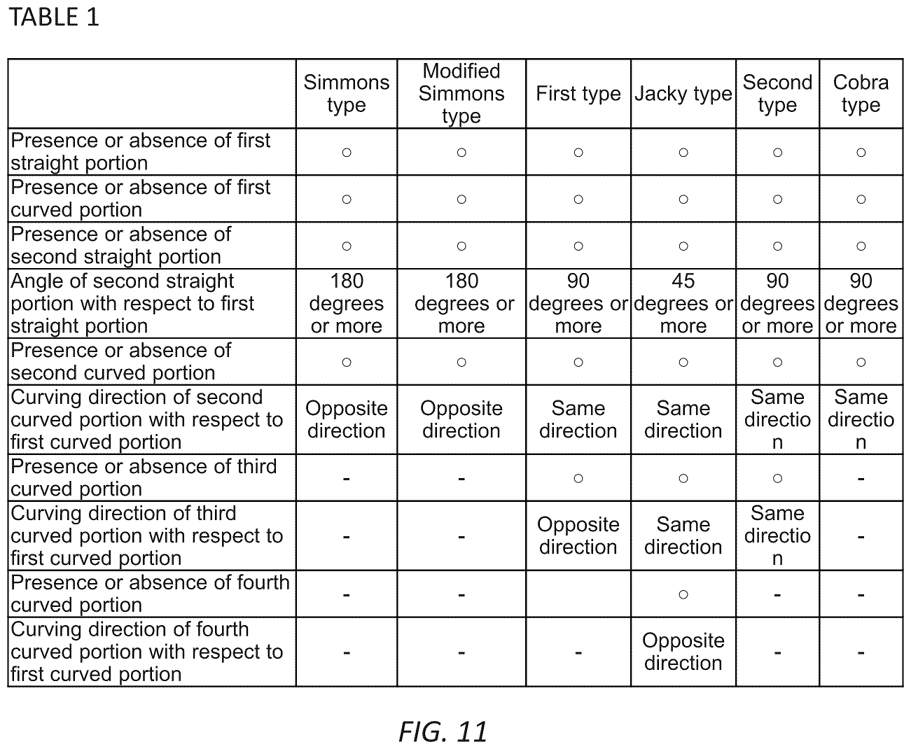

As shown in FIG. 10A, when the sheath 20, the hemostatic valve 70 (or the Y connector 80), and the dilator 40 are coupled to form an assembled state, the dilator tube body 41 is disposed in an inner cavity of the sheath shape-imparted portion 90. When a proximal end of the dilator distal end portion 45 is located on the proximal end side with respect to a proximal end of the sheath shape-imparted portion 90, the sheath shape-imparted portion 90 is deformed straight by the dilator distal end portion 45. That is, the dilator distal end portion 45 is more flexible than the dilator main body portion 44, but has such a hardness that the sheath shape-imparted portion 90 can be deformed straight.

As in a modification shown in FIG. 10B, when the proximal end of the dilator distal end portion 45 is located on the distal end side with respect to a distal end of the sheath shape-imparted portion 90, the sheath shape-imparted portion 90 is deformed straight by the dilator main body portion 44 which is located on the proximal end side of the dilator distal end portion 45 and is harder than the dilator distal end portion 45.

As in another modification shown in FIG. 10C, the dilator tube body 41 may be formed of a uniform material over the entire length, and may have a constant hardness over the entire length except for the tapered portion 45A. In this case, the sheath shape-imparted portion 90 is deformed straight by the dilator tube body 41 having a constant hardness.

Next, an example of a method of using the guiding sheath 10 according to the third embodiment will be described. Here, as shown in FIG. 6, a case where the guiding sheath 10 is inserted from the right radial artery A1 and indwelt in the left common carotid artery A4 will be described as an example. The surgeon may cause the guiding sheath 10 accessed from the right radial artery A1 to reach the right common carotid artery A5, or may cause the guiding sheath 10 accessed from the left radial artery to reach the left common carotid artery A4 or the right common carotid artery A5.

First, before the guiding sheath 10 is introduced into the blood vessel, as shown in FIGS. 10A to 10C, the sheath 20, the hemostatic valve 70, and the dilator 40 are coupled to form an assembled state. At this time, the sheath shape-imparted portion 90 is deformed straight by the dilator tube body 41 disposed in the inner cavity. By coupling the sheath 20, the hemostatic valve 70, and the dilator 40, the surgeon can integrally operate the sheath 20, the hemostatic valve 70, and the dilator 40 when inserting the guiding sheath 10 into the blood vessel.

Next, by a method similar to that of the first embodiment, the surgeon punctures the radial artery A1, inserts the guide wire 60 into the blood vessel from the radial artery A1, and inserts the guiding sheath 10 into the blood vessel along the guide wire 60 while advancing the guide wire 60. Since the sheath shape-imparted portion 90 is linear due to the rigidity of the dilator tube body 41, the sheath shape-imparted portion 90 is easily inserted into the blood vessel.

Next, the surgeon causes the guiding sheath 10 in the assembled state to reach the aortic arch A3 from the right subclavian artery A2. At this time, the surgeon keeps the guide wire 60 in the vicinity of the ascending aorta such that a blood vessel perforation does not occur. When the guiding sheath 10 reaches the meandering portion of the ascending aorta, the surgeon releases the coupling between the hemostatic valve 70 and the dilator 40, slightly pulls back the dilator 40, and disposes the distal end of the dilator 40 on the proximal end side of the sheath shape-imparted portion 90. Further, the surgeon disposes the flexible distal end portion of the guide wire 60 inside the sheath shape-imparted portion 90. Accordingly, the shape of the sheath shape-imparted portion 90 can be restored. Next, the surgeon applies a torque to the guiding sheath 10 to engage the sheath shape-imparted portion 90 with the left common carotid artery A4.

Next, the surgeon advances the guiding sheath 10 along the left common carotid artery A4. When the distal end of the guiding sheath 10 reaches the vicinity of a target, the surgeon stops pushing the guiding sheath 10. Next, the surgeon leaves the sheath 20 in the blood vessel and removes the guide wire 60 and the dilator 40. When the dilator 40 is removed, the valve body 71 in the housing 72 of the hemostatic valve 70 is closed to prevent backflow of blood. Accordingly, the sheath 20 and the hemostatic valve 70 can be used to perform a procedure of inserting a medical instrument corresponding to a treatment site to perform diagnosis or treatment.

As described above, the guiding sheath 10 of an aspect (1) according to the embodiment is the guiding sheath 10 capable of reaching a carotid artery from a radial artery. The guiding sheath 10 includes: the dilator 40 in which an inner cavity, through which the guide wire 60 is insertable is provided, over an entire length of the dilator 40 and which includes the dilator distal end portion 45 and the dilator main body portion 44; and the sheath 20 which covers outside of the dilator 40 and includes the sheath distal end portion 24 and the sheath main body portion 25. The sheath distal end portion 24 is disposed on the distal end side of the sheath main body portion 25 and is formed of a material more flexible than a material forming the sheath main body portion 25. At least one of the dilator distal end portion 45 and the sheath distal end portion 24 includes a shape-imparted portion to which a shape is imparted in advance to facilitate insertion into the carotid artery. Accordingly, the guiding sheath 10 has a high torque transmission performance because the sheath main body portion 25 is hard, and can effectively reduce a risk of a blood vessel perforation because the sheath distal end portion 24 is flexible. Since the guiding sheath 10 includes the shape-imparted portion on at least one of the dilator distal end portion 45 and the sheath distal end portion 24, the surgeon can integrally operate the dilator 40 and the sheath 20 to cause the dilator 40 and the sheath 20 to reach the carotid artery from the radial artery through the aortic arch or the subclavian artery. Therefore, in the guiding sheath 10, the dilator 40 and the sheath 20 integrally reach the carotid artery which is accessed from the radial artery and communicates with a target treatment site, and the sheath 20 can be easily indwelt in the carotid artery that communicates with the target treatment site.

The guiding sheath 10 of an aspect (2) is the guiding sheath 10 according to the aspect (1). The dilator distal end portion 45 is imparted with a shape in advance to facilitate insertion into the carotid artery when the dilator distal end portion 45 is inserted into the aortic arch. Accordingly, the guiding sheath 10 can easily reach the carotid artery from the radial artery through the aortic arch by the dilator 40 having a shape easy to be inserted into the carotid artery.

The guiding sheath 10 of an aspect (3) is the guiding sheath 10 according to the aspect (1) or (2). The reinforcement body 34 is embedded in the sheath 20 from the sheath distal end portion 24 to the sheath main body portion 25, and a proximal end of the reinforcement body 34 is disposed on the distal end side of a proximal end of the sheath main body portion 25. Accordingly, since the reinforcement body 34 is not disposed on the proximal end portion of the sheath main body portion 25, it is possible to reduce a thickness and a diameter of the proximal end portion of the sheath main body portion 25. Therefore, a burden on a puncture site of an arm of a patient can be reduced, and a hemostatic time can be reduced.

The guiding sheath 10 of an aspect (4) is the guiding sheath 10 according to the aspect (3). The reinforcement body 34 is a braided tube obtained by braiding a plurality of metal wires into a tubular shape, a coil obtained by spirally winding at least one metal wire, or a metal pipe in which at least one slit is formed.

The guiding sheath 10 of an aspect (5) is the guiding sheath 10 according to any one of the aspects (1) to (4). An outer diameter of the sheath main body portion 25 is smaller than an outer diameter of the sheath distal end portion 24. Accordingly, a burden on a puncture site of an arm of a patient can be reduced, and a hemostatic time can be reduced.

The guiding sheath 10 of an aspect (6) is the guiding sheath 10 according to any one of the aspects (1) to (5). The sheath main body portion 25 includes the sheath proximal end portion 28 disposed on the proximal end side of the sheath main body portion 25 and the sheath physical property gradient portion 29 disposed between the sheath proximal end portion 28 and the sheath distal end portion 24. In the sheath physical property gradient portion 29, a blending ratio of a material forming the sheath distal end portion 24 and a material forming the sheath proximal end portion 28 gradually changes from the proximal end side to the distal end side. Accordingly, the sheath physical property gradient portion 29 whose hardness gradually decreases from the proximal end side toward the distal end side can be implemented by a stable integral structure.

The guiding sheath 10 of an aspect (7) is the guiding sheath 10 according to any one of the aspects (1) to (6). The dilator main body portion 44 includes the dilator proximal end portion 48 disposed on the proximal end side of the dilator main body portion 44 and the dilator physical property gradient portion 49 disposed between the dilator proximal end portion 48 and the dilator distal end portion 45. In the dilator physical property gradient portion 49, a blending ratio of a material forming the dilator distal end portion 45 and a material forming the dilator proximal end portion 48 gradually changes from the proximal end side to the distal end side. Accordingly, the dilator physical property gradient portion 49 whose hardness gradually decreases from the proximal end side toward the distal end side can be implemented by a stable integral structure.

The guiding sheath 10 of an aspect (8) is the guiding sheath 10 according to any one of the aspects (1) to (7). The shape-imparted portion is provided in the sheath 20. Accordingly, by using the sheath shape-imparted portion 90, the surgeon can integrally cause the sheath 20 to reach the carotid artery from the radial artery through the aortic arch or the subclavian artery.

The guiding sheath 10 of an aspect (9) is the guiding sheath 10 according to any one of the aspects (1) to (8). When the sheath 20 and the dilator 40 are assembled, the shape of the shape-imparted portion is corrected to a substantially straight shape by the dilator 40. Accordingly, in a state where the sheath 20 and the dilator 40 are assembled, the sheath 20 and the dilator 40 can easily reach the carotid artery or the vicinity of the carotid artery.

The guiding sheath 10 of an aspect (10) is the guiding sheath 10 according to any one of aspects (1) to (9). The shape-imparted portion includes the first straight portion 51, the first curved portion 52 on the distal end side of the first straight portion 51, the second straight portion 53 on the distal end side of the first curved portion 52, and the second curved portion 54 on the distal end side of the second straight portion 53. An angle of the second straight portion 53 with respect to the first straight portion 51 is 45 degrees or more. Accordingly, in a state where the sheath 20 and the dilator 40 are assembled, the surgeon can easily cause the sheath 20 and the dilator 40 to reach the carotid artery or the vicinity of the carotid artery.

The guiding sheath 10 of an aspect (11) is the guiding sheath 10 according to the aspect (10). The shape of the shape-imparted portion is a Simmons type. Accordingly, by using the shape-imparted portion having a specific shape of the guiding sheath 10, the surgeon can cause the shape-imparted portion to reach the carotid artery from the radial artery through the aortic arch or the subclavian artery.

The method in the embodiment described above may be a method of indwelling the sheath 20 in the left common carotid artery A4 from the right radial artery A1 through the aortic arch A3, a method of indwelling the sheath 20 in the right common carotid artery A5 from the right radial artery A1 through the right subclavian artery A2, a method of indwelling the sheath 20 in the left common carotid artery A4 from the left radial artery through the aortic arch A3, or a method of indwelling the sheath 20 in the right common carotid artery A5 from the left radial artery through the aortic arch A3 and a brachiocephalic artery A6. That is, the method in the embodiment described above is a method of indwelling the sheath 20 for causing a device for a cerebral vascular treatment to reach a target site in the carotid artery (the left common carotid artery A4 or the right common carotid artery A5). The guiding sheath 10 includes: the dilator 40 in which an inner cavity, through which the guide wire 60 is insertable is provided, over an entire length of the dilator 40 and which includes the dilator main body portion 44 and the dilator distal end portion 45 which is disposed on the distal end side of the dilator main body portion 44 and more flexible than the dilator main body portion 44; and the sheath 20 which covers outside of the dilator 40 and includes the sheath main body portion 25 and the sheath distal end portion 24 which is disposed on the distal end side of the sheath main body portion 25. The method may include: inserting the guiding sheath 10 into the artery from the radial artery (the right radial artery A1 or the left radial artery) while advancing the guide wire 60, and causing the guiding sheath 10 to reach the aortic arch A3, the right subclavian artery A2, or the brachiocephalic artery A6; pulling back the guide wire 60 to the proximal end side with respect to the guiding sheath 10 and partially restoring the shape of the shape-imparted portion 90 to which the shape is imparted in advance on at least one of the dilator distal end portion 45 and the sheath distal end portion 24; causing the guiding sheath 10 to reach the carotid artery (the left common carotid artery A4 or the right common carotid artery A5); and removing the guide wire 60 and the dilator 40 from the artery while leaving the sheath 20. Accordingly, the surgeon can operate the sheath main body portion 25 having a high torque transmission performance, and integrally operate the dilator 40 and the sheath 20 while reducing the risk of the blood vessel perforation by the flexible sheath distal end portion 24, thereby causing the shape-imparted portion 90 of at least one of the dilator distal end portion 45 and the sheath distal end portion 24 to reach the carotid artery from the radial artery. Therefore, in the method, the sheath 20 can be easily indwelt in the carotid artery which is accessed from the radial artery and communicates with the target treatment site.

The disclosure is not limited to the above-described embodiments, and various modifications can be made to those skilled in the art within the technical idea of the invention. For example, both the sheath tube body 21 and the dilator tube body 41 may have the shape-imparted portion.

The detailed description above describes embodiments of a guiding sheath including a sheath and a dilator inserted into an inner cavity of the sheath. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A guiding sheath configured to reach a carotid artery from a radial artery, the guiding sheath comprising:
a dilator, the dilator including an inner cavity configured to receive a guide wire, the guide wire being is insertable over an entire length of the dilator and which includes a dilator distal end portion and a dilator main body portion;
a sheath which covers outside of the dilator and includes a sheath distal end portion and a sheath main body portion;
the sheath distal end portion is disposed on a distal end side of the sheath main body portion and is formed of a material more flexible than a material forming the sheath main body portion; and
at least one of the dilator distal end portion and the sheath distal end portion includes a shape-imparted portion to which a shape is imparted in advance to facilitate insertion into the carotid artery.

2. The guiding sheath according to claim 1, wherein
the dilator distal end portion is imparted with the shape in advance to facilitate insertion into the carotid artery when the dilator distal end portion is inserted into an aortic arch.

3. The guiding sheath according to claim 1, wherein
a reinforcement body is embedded in the sheath from the sheath distal end portion to the sheath main body portion, and a proximal end of the reinforcement body is disposed on a distal end side of a proximal end of the sheath main body portion.

4. The guiding sheath according to claim 3, wherein
the reinforcement body is a braided tube obtained by braiding a plurality of metal wires into a tubular shape, a coil obtained by spirally winding at least one metal wire, or a metal pipe in which at least one slit is formed.

5. The guiding sheath according to claim 1, wherein
an outer diameter of the sheath main body portion is smaller than an outer diameter of the sheath distal end portion.

6. The guiding sheath according to claim 1, wherein
the sheath main body portion includes a sheath proximal end portion disposed on a proximal end side of the sheath main body portion and a sheath physical property gradient portion disposed between the sheath proximal end portion and the sheath distal end portion; and
in the sheath physical property gradient portion, a blending ratio of a material forming the sheath distal end portion and a material forming the sheath proximal end portion gradually changes from the proximal end side to the distal end side.

7. The guiding sheath according to claim 6, wherein
the dilator main body portion includes a dilator proximal end portion disposed on a proximal end side of the dilator main body portion and a dilator physical property gradient portion disposed between the dilator proximal end portion and the dilator distal end portion; and
in the dilator physical property gradient portion, a blending ratio of a material forming the dilator distal end portion and a material forming the dilator proximal end portion gradually changes from the proximal end side to the distal end side.

8. The guiding sheath according to claim 1, wherein
the shape-imparted portion is provided in the sheath.

9. The guiding sheath according to claim 8, wherein
when the sheath and the dilator are assembled, the shape of the shape-imparted portion is corrected to a substantially straight shape by the dilator.

10. The guiding sheath according to claim 1, wherein
the shape-imparted portion includes a first straight portion, a first curved portion on a distal end side of the first straight portion, a second straight portion on a distal end side of the first curved portion, and a second curved portion on a distal end side of the second straight portion; and
an angle of the second straight portion with respect to the first straight portion is 45 degrees or more.

11. The guiding sheath according to claim 10, wherein
the shape of the shape-imparted portion is a Simmons type.

12. A guiding sheath, the guiding sheath comprising:
a dilator, the dilator including an inner cavity configured to receive a guide wire, the dilator including a dilator distal end portion and a dilator main body portion;
a sheath configured to cover outside of the dilator and includes a sheath distal end portion and a sheath main body portion;
the sheath distal end portion being disposed on a distal end side of the sheath main body portion; and
at least one of the dilator distal end portion and the sheath distal end portion includes a shape-imparted portion configured to be imparted with a shape that facilitates insertion into a carotid artery.

13. The guiding sheath according to claim 12, wherein
the sheath distal end portion is formed of a material more flexible than a material forming the sheath main body portion;
the dilator distal end portion is imparted with the shape in advance to facilitate insertion into the carotid artery when the dilator distal end portion is inserted into an aortic arch; and
a reinforcement body is embedded in the sheath from the sheath distal end portion to the sheath main body portion, and a proximal end of the reinforcement body is disposed on a distal end side of a proximal end of the sheath main body portion.

14. The guiding sheath according to claim 13, wherein
the reinforcement body is a braided tube obtained by braiding a plurality of metal wires into a tubular shape, a coil obtained by spirally winding at least one metal wire, or a metal pipe in which at least one slit is formed.

15. The guiding sheath according to claim 12, wherein
an outer diameter of the sheath main body portion is smaller than an outer diameter of the sheath distal end portion.

16. A guiding sheath, the guiding sheath comprising:
a dilator, the dilator including an inner cavity configured to receive a guide wire, the dilator including a dilator distal end portion and a dilator main body portion;
a sheath which covers outside of the dilator and includes a sheath distal end portion and a sheath main body portion;
the sheath distal end portion is disposed on a distal end side of the sheath main body portion and is formed of a material more flexible than a material forming the sheath main body portion;
at least one of the dilator distal end portion and the sheath distal end portion includes a shape-imparted portion; and
a reinforcement body is embedded in the sheath from the sheath distal end portion to the sheath main body portion, and a proximal end of the reinforcement body is disposed on a distal end side of a proximal end of the sheath main body portion.

17. The guiding sheath according to claim 16, wherein
the reinforcement body is a braided tube obtained by braiding a plurality of metal wires into a tubular shape, a coil obtained by spirally winding at least one metal wire, or a metal pipe in which at least one slit is formed.

18. The guiding sheath according to claim 16, wherein
an outer diameter of the sheath main body portion is smaller than an outer diameter of the sheath distal end portion.

19. The guiding sheath according to claim 16, wherein
the sheath main body portion includes a sheath proximal end portion disposed on a proximal end side of the sheath main body portion and a sheath physical property gradient portion disposed between the sheath proximal end portion and the sheath distal end portion;
in the sheath physical property gradient portion, a blending ratio of a material forming the sheath distal end portion and a material forming the sheath proximal end portion gradually changes from the proximal end side to the distal end side;
the dilator main body portion includes a dilator proximal end portion disposed on a proximal end side of the dilator main body portion and a dilator physical property gradient portion disposed between the dilator proximal end portion and the dilator distal end portion; and
in the dilator physical property gradient portion, a blending ratio of a material forming the dilator distal end portion and a material forming the dilator proximal end portion gradually changes from the proximal end side to the distal end side.

20. A method of indwelling a guiding sheath according to claim 1, the method comprising:
inserting the guide wire into an artery from the radial artery;
inserting the guiding sheath into the artery from the radial artery while advancing the guide wire;
causing the guiding sheath to reach one of an aortic arch, a right subclavian artery, or a brachiocephalic artery;
pulling back the guide wire to a proximal end side with respect to the guiding sheath and partially restoring the shape of the shape-imparted portion to which the shape is imparted in advance on the at least one of the dilator distal end portion and the sheath distal end portion;
causing the guiding sheath to reach one of a left common carotid artery or a right common carotid artery; and
removing the guide wire and the dilator from the artery while leaving the sheath in the one of the left common carotid artery and the right common carotid artery.
